(19) United States
(12) Patent Application Publication
(10) Pub. No.: US 2003/0018291 A1
(43) Pub. Date: Jan. 23, 2003

(54) Ear tube and method of insertion

(76) Inventors: Frank C. Hill, Columbia, SC (US); Tony Prescott, Bartlett, TN (US)

Correspondence Address: NEXSEN PRUET JACOBS & POLLARD, LLC, POST OFFICE DRAWER 2426, COLUMBIA, SC, 29202-2426, US

(21) Appl. No.: 10/197,977

(22) Filed: Jul. 18, 2002

Related U.S. Application Data
(63) Continuation-in-part of application No. 09/452,863, filed on Dec. 8, 1999.

(51) Int. Cl. A61M 5/00; A61F 11/00

(52) U.S. Cl. 604/8; 606/109

(57) ABSTRACT

An ear tube for draining and ventilating the middle ear that prevents perforation of the ear drum. The tube comprises a tubular stem having a lumen formed in it and a pair of ports formed in its walls for providing access to the lumen from the sides. A plurality of resilient arms extend from one end of stem at an acute angle to the stem and each other. The tube further comprises a collar that closes the arms against each other so that the ear tube can be slipped through an incision in the tympanic membrane. Once through the membrane, the arms resiliently spring open to anchor the tube. Preferably, the stem is formed from a translucent material and the collar is made of a transparent material so obstructions in the stem can be seen while the arms are formed from an opaque material so that a physician could easily see the arms through the tympanic membrane.

### CROSS REFERENCE TO RELATED APPLICATIONS

The inventor claims the priority benefit of utility application Ser. No. 09/452,863 filed Dec. 8, 1999.

### STATEMENT REGARDING FEDERALLY SPONSORED RESEARCH OR DEVELOPMENT

Not applicable.

### REFERENCE TO A SEQUENCE LISTING, A TABLE, OR A COMPUTER PROGRAM LISTING COMPACT DISK APPENDIX

Not applicable.

### BACKGROUND OF THE INVENTION

The present invention relates to ear tubes. In particular, the present invention relates to tubes for draining fluid through the ear drum and methods of inserting the tubes.

Typically, pressure within the middle ear is regulated by the Eustachian tube. Whenever the pressure within the middle ear is greater or less than the surrounding atmosphere, the Eustachian tube opens to equalize pressure. If the Eustachian tube is not functioning properly, the pressure within the middle ear tends to drop. The negative pressure in the middle ear draws fluid from the surrounding tissue and causes fluid to accumulate in the middle ear. This fluid causes swelling and infections within the middle ear.

In order to drain and ventilate the middle ear, ear tubes can be used. The tubes equalize pressure with the surrounding atmosphere, thereby eliminating the accumulation of fluid and reducing the risk of infection. To install a tube, a small incision is cut in the tympanic membrane, commonly called the ear drum, and the tube is inserted through the incision.

There are various types of ear tubes. One type of tube is formed as a shank with a trailing flange. This type of tube tends to fall out of the ear after a short period of time. Another type of tube, commonly called a T-tube on account of its shape, rarely falls out spontaneously, but requires removal by a physician. The T-tube has a flange that resiliently opens upon insertion through the ear drum, much like a toggle bolt. Unfortunately, the T-tube suffers from several disadvantages. The T-tube tends to leave a perforation in the eardrum about 25% of the time, which is considerably more than other types of tubes. The flange of the T-tube unfolds at an angle of 90° to the axis of the tube. These arms tend to “tent up” the eardrum and erode through the ear drum, causing a perforation. The reason for this “tenting-up” is that the eardrum is not flat, as sometimes thought, but has a complex curved shape such that the shape of a conventional T-tube puts undo pressure on the eardrum. Moreover, the T-tubes tend to shift and align themselves across the ear canal or block the tube, thereby making it difficult to see down the lumen of the tube to determine if the tube is obstructed. Additionally, the T-tubes tend to become obstructed with a plug of desiccated fluid or blood.

Yet another disadvantage of these T-tubes is the need for special instruments for inserting the tubes. During the insertion of the T-tubes, instruments are required to keep the arms of the T-tubes in alignment before the tube is pushed through the tympanic membrane. The insertion instruments are often difficult to operate and can cause the overall draining process to be both lengthy and tedious.

Therefore, there is a need for an improved ear tube that can be inserted without the use of special instruments and that stays in place unless removed, but does not tend to cause permanent perforations in the ear drum and that stays perpendicular to the ear drum and that can be readily cleared if it becomes obstructed.

### SUMMARY OF THE INVENTION

According to its major aspects and broadly stated, the present invention is an ear tube for draining and ventilating the middle ear and method of insertion. In a first embodiment, the ear tube includes a cylindrical stem having a lumen longitudinally formed therein and at least one port extending through the surface of the stem for providing access to the lumen. Three or four arms extend from one end of the stem. These arms can be folded to fit within an envelop defined by the outer dimensions of the stem and then resiliently spring open once through the tympanic membrane to anchor the tube. The arms are angled, preferably approximately 60° from the axis of the tube when open. Preferably, the stem is made from a translucent material so that one can see if it is plugged, while the arms are formed from a colored, opaque material so that a physician could easily see the arms through the translucent tympanic membrane.

An alternative second embodiment of the ear tube includes a cylindrical collar having a handle that surrounds a cylindrical stem having a lumen longitudinally formed therein and at least one port extending through the surface of the stem for providing access to the lumen. The collar is slidably carried by the stem. As with the previously described embodiment, three or four arms extend from one end of the stem. These arms can be folded to fit within an envelop defined by the outer dimensions of the stem and then resiliently spring open after having passed through the tympanic membrane to anchor the tube. In operation, the collar keeps the arms folded and in alignment until the arms of the ear tube are pushed through the tympanic membrane. After the arms have been pushed through the membrane, the arms resiliently spring open so as to anchor the ear tube to the tympanic membrane. A flange is further provided at the end of the collar to reduce the impact of the forward motion of the collar on the tympanic membrane. Preferably, the collar is made of a translucent material so that one can see what is taking place, including the pushing of the arms through the tympanic membrane.

The present invention further includes a method for inserting the ear tube. The method includes the following steps: 1) providing an ear tube having a stem, arms extending radially from one end of the stem, and a collar surrounding the stem that holds the arms in alignment and that is moveable along the length of the stem; 2) placing the ear tube within an ear so that the end of the collar is next to the tympanic membrane of the ear; 3) pushing the arms of the ear tube through the tympanic membrane; and 4) removing the collar from the ear tube.

A major feature of the present invention is the use of an ear tube collar that holds the arms of the ear tube in alignment during the insertion of the ear tube through the tympanic membrane. Typically, special instruments must be employed to insert ear tubes having flanges that resiliently spring open. The ear tube collar of the present invention provides a simple and effective alternative of keeping the flanges or arms of these ear tubes in alignment during their insertion within the tympanic membrane. Further, a flange is provided at the end of the collar to reduce the impact of the forward motion of the collar on the tympanic membrane.

Another feature of the present invention is the optical characteristics of the ear tube. Both the stem and the collar are made from a translucent material. As a result of this feature, the physician can more easily detect any obstructions clogging the lumen of the tube. Further, the physician can view the positioning of the arms as they are being pushed through the collar and into the tympanic membrane. Additionally, the arms of the ear tube are made of an opaque material so that they can be detected through the tympanic membrane. This feature allows for easier installation of the tube since the physician can detect the positioning of the arms through the tympanic membrane.

Still another feature of the present invention is the use of at least three angled arms anchoring the tube within the tympanic membrane. As a result of the angled configuration of the arms, the incidence of perforations in the tympanic membrane is greatly reduced. Further, the number of arms contributes to the stability of the ear tube within the tympanic membrane.

The arm configuration that withstands rotation or shifting within the tympanic membrane is another important feature of the present invention. Consequently, the tube will not be blocked by becoming mispositioned with the end against the ear canal. The physician can look down the lumen or through the translucent wall of the tube to detect any obstructions.

The ability to clear any obstructions from the tube in situ is an important advantage of the present invention. This advantage is enabled by the ports formed in the stem of the tube, which allow a physician easy access to remove any desiccated plug or blood clot by grasping it through the ports, without requiring removal or replacement of the tube.

Other features and advantages of the present invention will be apparent to those skilled in the art from a careful reading of the Detailed Description of a Preferred Embodiment presented below and accompanied by the drawings.

### BRIEF DESCRIPTION OF THE DRAWINGS

FIG. 1 is a side, partially cross sectional view of the tube installed within the tympanic membrane, according to a preferred embodiment of the resent invention;

FIG. 2 is a rear view of the tube, according to a preferred embodiment of the present invention; and

FIG. 3 is a perspective view of the tube, according to a preferred embodiment of the present invention.

FIG. 4 is a side, partially cross sectional view of the tube having a collar holding the arms of the tube in an aligned position, according to an alternative preferred embodiment of the present invention;

FIG. 5 is a side, partially cross sectional view of the tube being installed within the tympanic membrane, according to an alternative preferred embodiment of the present invention;

FIG. 6 is a side, partially cross sectional view of the tube installed within the tympanic membrane, according to an alternative preferred embodiment of the present invention; and

FIG. 7 is a cross sectional of the tube and the collar of the tube, according to an alternative preferred embodiment of the present invention.

### DETAILED DESCRIPTION OF A PREFERRED EMBODIMENT

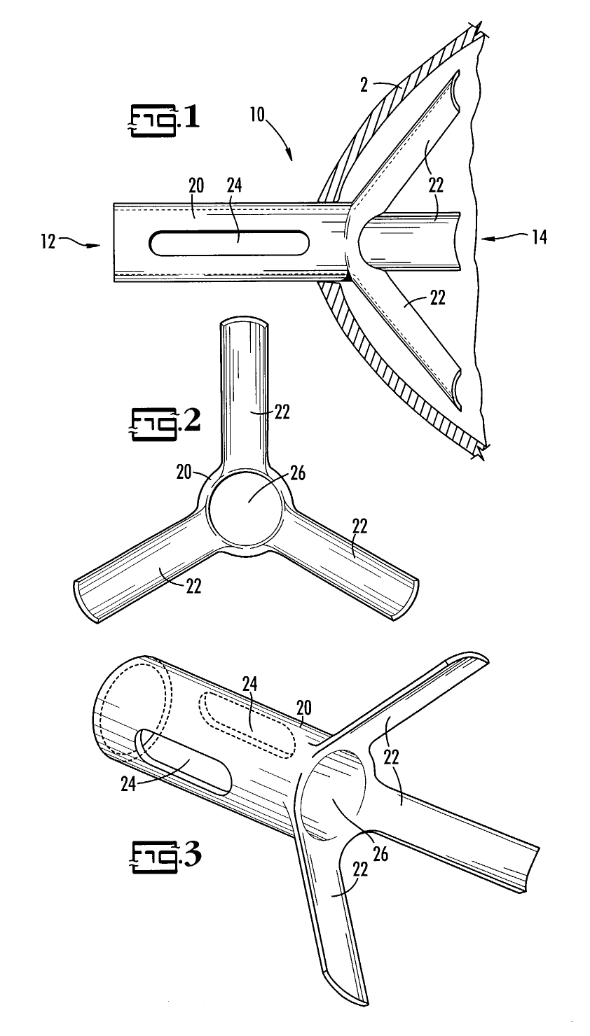

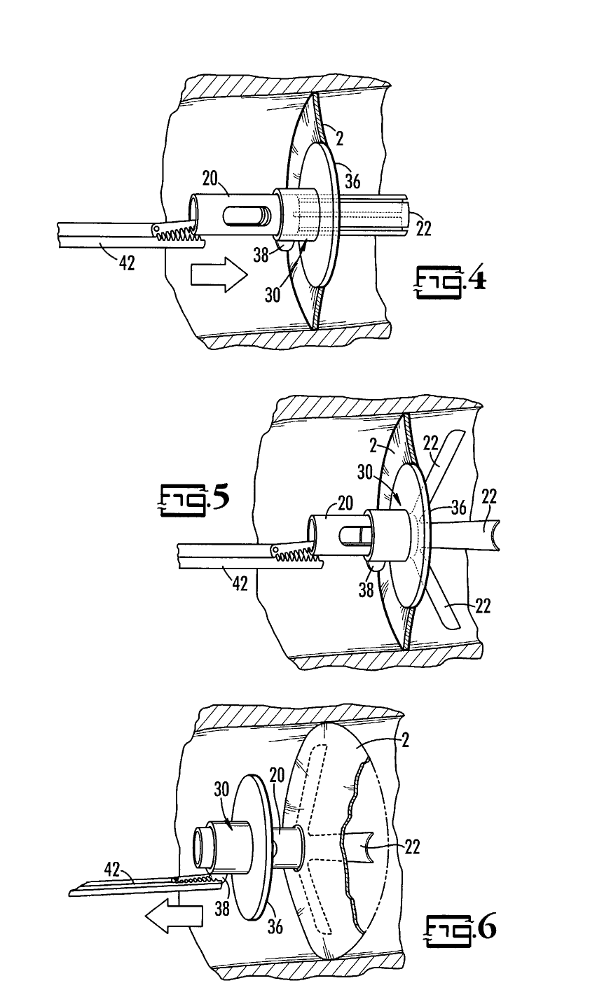

Referring now to the figures, the present invention is an ear tube for draining and ventilating the middle ear. According to a preferred embodiment, the tube, generally referred to by reference number 10, comprises a tubular stem 20 having a lumen 26 longitudinally formed therein and at least one port 24 formed in the wall of the stem 20 for providing access to the lumen 26. By the term lumen, it is meant an interior space of tubular stem 20. A plurality of arms 22 extend from one end of stem 20 to anchor tube 10 within the tympanic membrane 2 subsequent to installation as illustrated by FIG. 1. For purposes of orientation in referring to the drawings, tube 10 has a front end 12 and a rear end 14.

Stem 20 is preferably cylindrical in shape having sufficient dimensions to fit within an incision in tympanic membrane 2. Stem 20 has lumen 26 formed therein from one end of stem 20 to the other end of stem 20. Lumen 26 is of sufficient diameter to provide drainage and ventilation from the middle ear to the outer ear, and preferably has a diameter similar to that of ear tubes in use in the art. Stem 20 has at least one port 24 extending laterally therethrough, that is, perpendicular to the axis of stem 20. Port 24 is of sufficient area to allow a physician access to any obstruction that may be blocking lumen 26 of the tube 10 in situ.

As previously discussed, the optical characteristics of the tube 10 are a feature of the present invention. Preferably, stem 20 has two opposing ports 24 formed in the surface. Stem 20 preferably is also formed from a translucent material. This translucent material in conjunction with ports 24 will allow a physician easy access to detect and remove any obstructions blocking lumen 26 without removing or replacing tube 10.

As shown in FIG. 1, tube 10 has plural arms 22 extending from the rear end 14 of tube 10. Arms 22 are preferably integrally formed with stem 20 and at an angle A with respect to stem 20 of between 20° and 40° so that the physician can look down the axis of stem 20 while arms 22 anchor themselves against a tympanic membrane that is at an angle with respect to the ear canal. Arms 22 preferably also have sufficient flexibility to allow arms 22 to be closed together to within an envelop defined by the diameter of tube 10 in order to pass through a small incision in a tympanic membrane 2; i.e., no larger incision is needed to insert tube 20 because it has three arms 22. Arms 22 are also sufficiently resilient to provide an anchoring function upon insertion through tympanic membrane 2 as illustrated in FIG. 1. Preferably, tube 10 has three equally spaced arms 22 protruding from rear end 14 of tube 10 at approximately equal angles of approximately 60 degrees with respect to their common axis but almost any angle from almost 90 degrees down to just enough of a spread to hold tube 10 in place and prevent tube 10 from being released from tympanic membrane 2 and not cause frequent perforations.

In use, arms 22 have sufficient flexibility to be forced together so that tube 10 can pass through an incision in the tympanic membrane 2 and resiliency to spring apart once through the tympanic membrane 2. The color of arms 22 allow a physician to easily observe the position of arms 22 through the tympanic membrane 2. Upon passing through the incision, arms 22 resiliently open to anchor tube 10 from prematurely exiting from tympanic membrane 2. Lumen 26 in tube 10 equalizes pressure in the middle ear with that of the surrounding atmosphere; moreover, lumen 26 provides draining and ventilation of the middle ear in order to prevent further infection. If any blood or desiccated fluid clogs lumen 26 through the draining process, the translucent material that stem 20 is made of allows it to be seen; ports 24 provide easy access for removal of the obstructions.

As shown in FIGS. 4-7, the present invention further includes an alternative embodiment of the tube 10. Similar to the previously described embodiment, tube 10 has front end 12 and rear end 14 and includes tubular stem 20 having lumen 26 longitudinally formed therein and at least one port 24 formed in the wall of the stem 20 for providing access to the lumen 26. Plural arms 22 extend from rear end 14 of tube 10.

In addition to these previously described features, the tube 10 further includes a collar 30 that surrounds the stem 20 and that is used to hold plural arms 22 in alignment during the insertion of the tube within the tympanic membrane 2. By the term alignment, it is meant that arms 22 are kept folded so that they fit within an envelope defined by the outer dimensions of stem 20. As shown in FIG. 4, collar 30 is slidably carried by stem 20 and is preferably cylindrical in shape. FIG. 7 illustrates a cross sectional view of tube 10 and collar 30. As shown, collar 30 is dimensioned to receive tube 10 and preferably can be slid along the longitudinal length of tube 10. In particular, collar 30 is moveable so that when collar 30 engages tympanic membrane, arms 22 of tube will go forward and through the membrane 2 and collar 30 will remain outside the membrane 2. The dimensions of collar 30 are such that it will fit within the ear canal.

A particular feature of the present invention is the use of collar 30 in combination with tube 10. As previously stated, special instruments must typically be employed to insert ear tubes having flanges or arms that resiliently spring open. The collar 30 of the present invention provides a simple and effective alternative to keeping the flanges or arms of these ear tubes in alignment during their insertion within the tympanic membrane 2.

For purposes of orientation in referring to the drawings, collar 30 has a front end 32 and a rear end 34. Preferably, collar 30 includes a flange 36 at front end 32 and a handle 38 at rear end 34. As previously discussed, flange 36 is provided to reduce the impact of the forward motion of the tube 10 on the tympanic membrane 2. In particular, flange 36 is dimensioned so that when it engages the tympanic membrane 2, flange 36 distributes the pushing force exerted to insert the tube 10. Further, as arms 22 are pushed through the tympanic membrane, flange 36 helps to prevent collar 30 from also going forward and through the membrane 2. Handle 38 of collar 30 is provided to aid the physician in removing collar 30 once the tube 10 has been inserted within the tympanic membrane 2.

As illustrated by FIG. 4, arms 22 are preferably integrally formed with stem 20 and have sufficient flexibility to allow arms 22 to be closed together to within an envelope 40 defined by the diameter of tube 10 in order to pass through a small incision in the tympanic membrane 2.

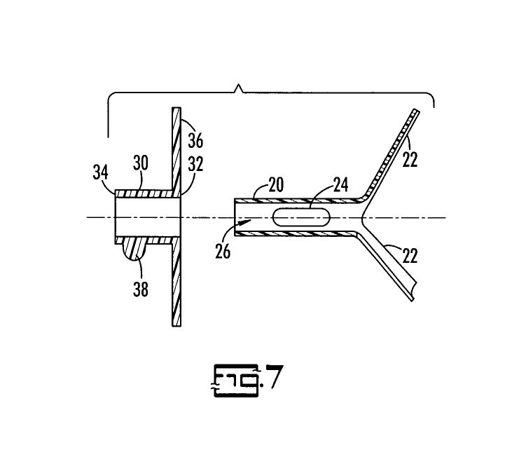

In operation, collar 30 holds plural arms 22 together when it is slid toward rear end 14 of stem 20. After arms 22 have passed through tympanic membrane 2, they are sufficiently resilient to spring open and anchor tube 10 within the membrane 2, as shown in FIG. 5. Collar 30 is thereafter removed by a means for removing 42 such as an alligator clip. As illustrated in FIG. 6, a physician merely engages handle 38 of collar 30 with removing means 42 and slides collar 30 down the length of the tube 10 towards front end 12 of stem 20 in order to remove collar 30.

As previously stated, the optical characteristics of ear tube 10 are a feature of the present invention. Preferably, collar 30 and stem 20 of tube 10 are made of translucent material. Further, arms 22 of tube are preferably made of a colored or opaque material. Accordingly, a physician can more easily detect any obstructions clogging the lumen 26 of the tube 10. Additionally, the physician can view the positioning of the arms 22 as they are being pushed through the collar 30 and into the tympanic membrane 2. This feature allows for easier and more accurate insertion of the tube 10 by the physician.

The present invention further includes a method for inserting the tube within the tympanic membrane 2. As depicted in FIG. 4, tube 10 having collar 30 is first provided. Next, the tube 10 is inserted within the tympanic membrane by placing tube 10 within the ear canal from the outer ear to tympanic membrane 2 and pushing arms 22 of tube 10 through an incision in the membrane 2 with a means for pushing 50 such as a clip. As shown, collar 30 holds arms of tube 10 in alignment as they are being inserted. FIG. 5 illustrates that once arms 22 are within the tympanic membrane 2, they resiliently spring open to anchor tube 10 to the membrane 2. At this point, collar 30 is removed by means for removing 42. Preferably, collar is removed by engaging handle 38 of collar 30 with removing means 42 and pulling collar 30 along the longitudinal length of tube 10 toward front end 12 of stem 20.

It will be apparent to those skilled in the art that many changes and substitutions can be made to the preferred embodiments herein described without departing from the spirit and scope of the present invention

What is claimed is:

1. An ear tube for draining and ventilating a tympanic membrane, said tube comprising:
a stem having a first end and an opposing second end, said first end having a lumen formed therein between said first end and said second end;
at least two arms attached to said first end so that said at least two arms extend radially; and
a collar slidably carried by said stem.

2. The ear tube as recited in claim 1, wherein said collar holds said at least two arms together when said collar is slid toward said first end.

3. The ear tube as recited in claim 1, wherein said collar includes a flange.

4. The ear tube as recited in claim 1, wherein said collar includes a handle.

5. The ear tube as recited in claim 1, wherein said collar is made of transparent material.

6. The ear tube as recited in claim 1, wherein said at least two arms are integrally attached to said stem.

7. The ear tube as recited in claim 1, wherein said at least two arms comprise three arms.

8. The ear tube as recited in claim 1, wherein said at least two arms are equally spaced about said first end of said stem.

9. The ear tube as recited in claim 1, wherein said at least two arms, when folded fit within an envelop defined by said stem.

10. The ear tube as recited in claim 1, wherein said at least two arms extend resiliently.

11. The ear tube as recited in claim 1, wherein said stem has at least one port extending therethrough between said first end and said second end.

12. The ear tube as recited in claim 1, wherein said lumen is dimensioned to facilitate drainage and ventilation from a middle ear to an outer ear.

13. An ear tube for draining and ventilating a tympanic membrane, said tube comprising:
a stem having a first end and an opposing second end, said first end having a lumen formed therein between said first end and said second end;
at least two arms attached to said first end so that said at least two arms extend radially, said at least two arms being foldable to fit within an envelop defined by the outer dimensions of said stem; and
a collar slidably carried by said stem, said collar holding said at least two arms within said envelop when said collar is slid to said first end.

14. The ear tube as recited in claim 14, wherein said collar includes a flange.

15. The ear tube as recited in claim 14, wherein said collar includes a handle.

16. The ear tube as recited in claim 14, wherein said collar is made of transparent material.

17. A method inserting an ear tube for draining and ventilating a tympanic membrane, comprising the steps of:
making an incision in said tympanic membrane;
providing an ear tube having a stem, plural arms extending radially from said stem, and a collar surrounding said plural arms and holding said plural arms in alignment; and
pushing said plural arms through said incision, said plural arms extending resiliently so that said arms spring apart when said arms pass through said tympanic membrane and said tympanic membrane resisting movement of said collar therethrough.

18. The method as recited in claim 20, further comprising the step of removing said collar from said ear tube.
